(19) United States
(12) Patent Application Publication
(10) Pub. No.: US 2021/0251801 A1
(43) Pub. Date: Aug. 19, 2021

(54) HANDPIECE FOR TREATMENT, TREATMENT DEVICE INCLUDING HANDPIECE, AND TREATMENT METHOD USING TREATMENT DEVICE

(71) Applicant: LUTRONIC CORPORATION, Goyang (KR)

(72) Inventor: Kwang Chon KO, Paju (KR)

(21) Appl. No.: 17/273,601

(22) PCT Filed: Jul. 22, 2019
(86) PCT No.: PCT/KR2019/009016

§ 371 Date: Mar. 4, 2021

(30) Foreign Application Priority Data
Sep. 6, 2018 (KR) 10-2018-0106356

(51) Int. Cl. A61F 7/00; A61B 18/14

(52) U.S. Cl. CPC A61F 7/00; A61B 18/1477; A61B 2018/00017; A61F 2007/0056; A61F 2007/0055

(57) ABSTRACT

A handpiece for treatment according to an embodiment of the present invention comprises: a housing having at least one through-hole formed in the tip end thereof; an insertion part that passes through the surface of tissue and is inserted into the tissue in a state in which at least a portion thereof is exposed through the through-hole; and a cooling channel for providing a cooling gas that is supplied to the surface of the tissue through the through-hole.

### TECHNICAL FIELD

The present disclosure relates to a handpiece for treatment, a treatment device including the same, and a treatment method using the same, and more particularly, to a handpiece for treatment inserted into a tissue of a human body to perform treatment in an invasive manner, a treatment device including the same, and a treatment method using the same.

### BACKGROUND ART

A method of treating a tissue may be classified into a method of treating a tissue from the outside the tissue and an invasive treatment method of performing treatment by inserting a part of or an entire treatment device into the tissue. In these methods, the invasive treatment method mainly uses a treatment device having a thin-necked insertion unit such as a needle or a catheter, and treatment is performed after the treatment device is inserted into a target position in the tissue.

The invasive treatment method includes various treatment behaviors, such as transferring a treating substance to the inside of tissue, performing surgical treatment through a mechanical operation while being adjacent to a specific tissue, or transferring energy to a target position in the tissue. Such a treatment method is disclosed in Korean Patent Application Publication No. 10-2011-0000790 or the like, and in addition to this, is applied in various ways.

In the invasive treatment method, a radio frequency (RF) treatment method, which transfers RF energy by inserting a part of or an entire RF electrode into the tissue, employs a principle in which once an RF current flows into the tissue through the electrode, and the tissue serves as resistance and generates heat energy.

However, when a temperature of a tissue excessively rises, a burn may be caused during treatment.

### DISCLOSURE

### Technical Problem

In order to solve the problem, an object of the present disclosure is to provide a treatment device or the like capable of preventing excessive temperature rise in a tissue during treatment.

Technical objects to be achieved by the present disclosure are not limited to the aforementioned technical objects, and other technical objects not described above may be evidently understood by a person having ordinary skill in the art to which the present disclosure pertains from the following description.

### Technical Solution

In order to solve the above problems, a handpiece for treatment according to an embodiment of the present disclosure includes: a housing having at least one through-hole formed in a tip end thereof; an insertion unit passing through a tissue surface and inserted into a tissue in a state in which at least a portion thereof is exposed through the through-hole; and a cooling channel providing a cooling gas supplied to the tissue surface through the through-hole.

In order to solve the above problems, a method of controlling a treatment device according to an embodiment of the present disclosure includes: positioning an insertion unit on a tissue surface; inserting at least a portion of the insertion unit into a tissue by passing through the tissue surface; and supplying a cooling gas to the tissue surface.

In order to solve the above problems, an RF treatment device according to an embodiment of the present disclosure includes: a main body including an RF generator and a refrigerant tank; and a handpiece connected to the main body, wherein the handpiece includes a housing having at least one through-hole formed in a tip end thereof; an insertion unit passing through a tissue surface and inserted into a tissue in a state in which at least a portion thereof is exposed through the through-hole, and applying RF energy transferred from the RF generator to an inside of the tissue; and a cooling channel providing a cooling gas transferred from the cooling tank to the tissue surface through the through-hole.

Other features of the present disclosure will be apparent from the following detailed description and the drawings.

### Advantageous Effects

In accordance with the embodiment of the present disclosure, an effect is at least as follows.

It is possible to prevent the excessive temperature rise in the tissue during treatment.

It should be noted that the effect of the present disclosure is not limited to that described above and other effects of the present disclosure are included in the following descriptions.

### DESCRIPTION OF DRAWINGS

FIG. 1 is a perspective view illustrating a radio frequency (RF) treatment device according to an embodiment of the present disclosure.

FIG. 2 is an exploded perspective view illustrating a handpiece of the RF treatment device in FIG. 1.

FIG. 3 is a block diagram illustrating a main control system of the RF treatment device in FIG. 1.

FIG. 4 is a cross-sectional view schematically illustrating an internal structure of a tip end portion of the handpiece in FIG. 2.

FIG. 5 is a flowchart illustrating a treatment method using the RF treatment device according to an embodiment of the present disclosure.

FIGS. 6 and 7 are views for explaining the treatment method using the RF treatment device according to an embodiment of the present disclosure.

FIG. 8 is a cross-sectional view schematically illustrating an internal structure of a tip end portion of the handpiece according to another embodiment of the present disclosure.

FIG. 9 is a flowchart illustrating a treatment method using the handpiece in FIG. 8.

FIGS. 10 and 11 are cross-sectional views schematically illustrating an internal structure of a tip end portion of the handpiece according to another embodiment of the present disclosure.

### MODE FOR DISCLOSURE

The merits and characteristics of the disclosure and a method of achieving the merits and characteristics will become more apparent from the embodiments described in detail in conjunction with the accompanying drawings. However, the disclosure is not limited to the disclosed embodiments, but may be implemented in different ways. The embodiments are provided to only complete the disclosure and to allow those skilled in the art to fully understand the category of the disclosure. The disclosure is defined by the category of the claims. The same reference numerals will be used to refer to the same or similar elements throughout the drawings.

Embodiments described herein will be described with reference to cross-sectional views and/or schematic views that are views of idealized embodiments of the present disclosure. Thus, the shape of the exemplary figures can be modified by manufacturing techniques and/or tolerances. Further, in the drawings of the present disclosure, each component may be somewhat enlarged or reduced in view of convenience of explanation. The same reference numerals will be used to refer to the same or similar elements throughout the drawings.

A “radio frequency (RF) treatment device” used herein includes all devices for treating animals such as mammals including humans. The treatment device may include various devices for performing treatment by transferring RF energy for the purpose of improving a condition of a lesion or tissue. In the following embodiments, a device for treating a skin lesion will be mainly described, but the embodiments are not limited thereto. Note that the present disclosure may be applied to various devices used to transfer the RF energy to various affected areas, including a device for surgically treating lesions within the body.

A “tissue” used herein means a set of cells forming various body organs of an animal including humans, and includes various tissues forming various organs within a body, including a skin tissue.

An ‘insertion unit’ herein means a component of the treatment device inserted into the tissue. Various components formed in a structure having a sharp, thin, and long end portion such as a needle, a microneedle, and a catheter, passing through a surface of the tissue, and inserted into the tissue, are included.

Hereinafter, the present disclosure will be described with reference to the drawings for explaining an RF treatment device according to embodiments of the present disclosure.

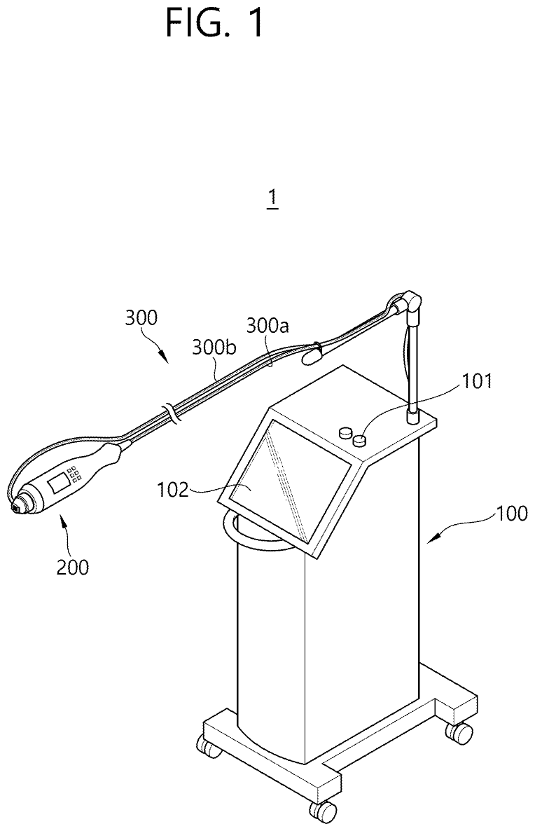

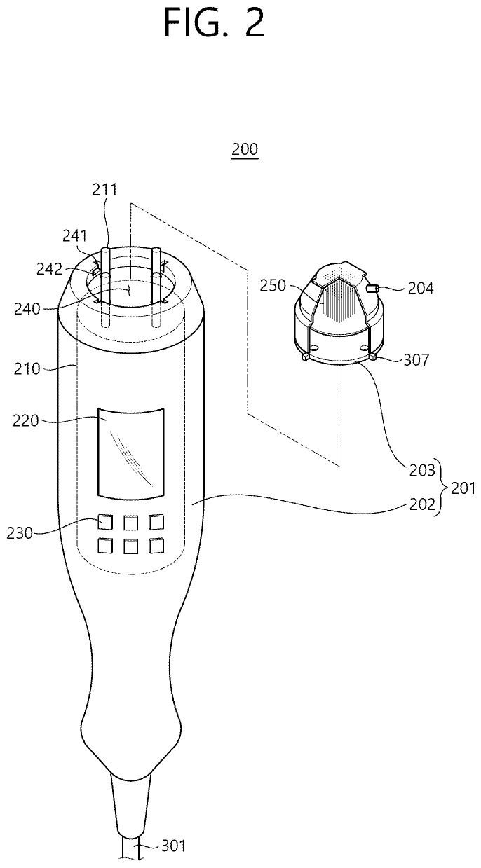

FIG. 1 is a perspective view illustrating a radio frequency (RF) treatment device according to an embodiment of the present disclosure, and FIG. 2 is an exploded perspective view illustrating a handpiece of the RF treatment device in FIG. 1.

As illustrated in FIG. 1, a medical RF device according to the present embodiment is composed of RF treatment devices, and such an RF treatment device 1 includes a main body 100, and a handpiece 200 capable of performing treatment while being gripped by a user.

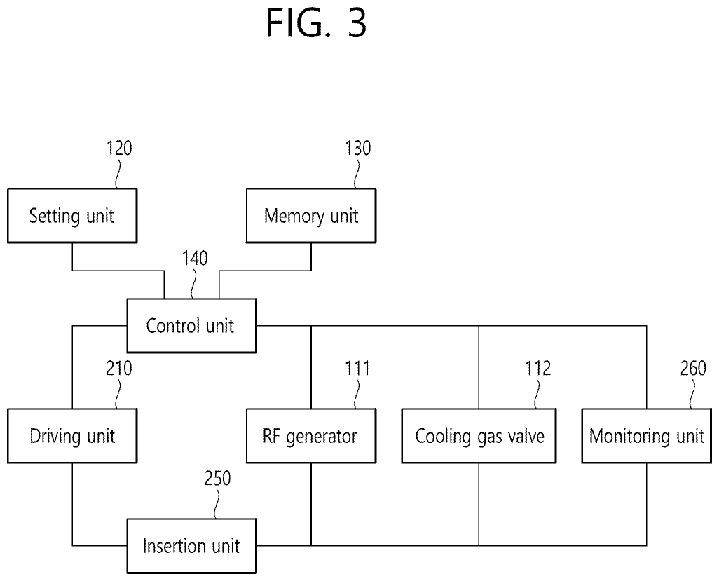

An RF generator 111 (see FIG. 3) is provided in the main body 100. The RF generator 111 generates RF energy used for treatment. The RF generator 111 generates RF energy to transfer it in a pulse form rather than a continuous waveform. The RF generator 111 may generate an RF pulse for various parameters (for example, output, pulse duration, pulse interval, frequency, and the like) according to a patient's physical constitution, a treatment purpose, a treatment site, or the like. The RF pulse generated by the RF generator of the present embodiment is an RF pulse for treatment, which is used for tissue treatment. The RF energy used for skin treatment may be adjusted in a range of 0.1 to 0.8 MHz.

Further, a refrigerant tank (not illustrated) is provided in the main body 100. The refrigerant tank stores, in a pressurized gas state or liquid state, a cooling gas provided to a surface of the tissue in which the RF energy is supplied to generate heat during a treatment process. As a refrigerant, cryogen and/or air may be used, and in addition to this, various gases, which are harmless to a skin and capable of decreasing heat of a skin, may be used.

A switch 101 for controlling operations of the treatment device, including turning on/off the switch and a display unit 102 displaying various information including operations of the treatment device may be provided on an outer surface of the main body 100. The display unit 102 may be composed of a touch screen to display various information and to directly set, by a user, details of the treatment through the display unit 102.

The handpiece 200 is connected to the main body by a connection unit 300. The connection unit 300 may transfer power, a control signal, or the like required for operations of various devices of the handpiece 200 from the main body 100. The connection unit 300 may be composed of cables including various signal lines, power lines, and the like, and may have a bending structure so as to be easily bent by the manipulation of the user.

The connection unit 300 may include a first line 300a for transferring the RF energy generated by the RF generator 111 to the handpiece 200, and a second line 300b transferring a cooling gas supplied from the refrigerant tank of the main body 100 to the handpiece 200.

As illustrated in FIG. 2, a housing 201 of the handpiece 200 may be separated from a handpiece body 202 and a tip 203.

A handpiece manipulation unit 230 and a handpiece display unit 220 may be provided on an outer surface of the handpiece body 202. The handpiece manipulation unit 230 may manipulate turning on/off of the handpiece 200, adjust an insertion depth of an insertion unit 250, or adjust a magnitude of energy transferred by the insertion unit 250. The handpiece display unit 220 may display, to the user, various information required in a setting mode or during the treatment. Therefore, the user may perform the treatment by manipulating the manipulation unit 230 while gripping the handpiece 200 in a hand, and easily grasp details of the treatment through the display unit 220.

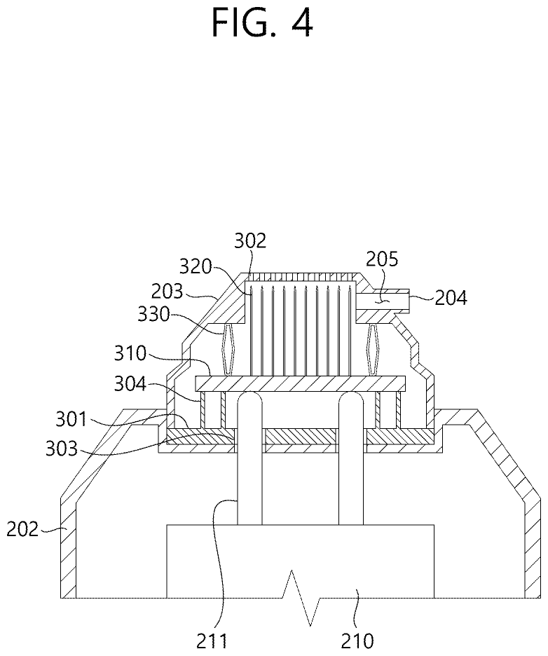

A driving unit 210 is installed in the handpiece 200. The driving unit 210 may move the insertion unit 250 to be optionally inserted into the tissue. The driving unit 210 may be formed using various linear actuators such as solenoids and oil/air cylinders, and linear motors, and the like. For example, the driving unit of the present embodiment linearly moves an output terminal 211 provided at one end thereof in a longitudinal direction. A plurality of needles 320 (see FIG. 4) corresponding to the insertion unit 250 are arranged at an end portion of the output terminal 211, and as the output terminal is moved linearly, the insertion unit 250 appears at one end of the handpiece 200 (one end in contact with a treatment position). As such, the insertion unit 250 may be inserted into or withdrawn from the tissue of a patient while moving forward/backward by driving of the driving unit 210.

As described above, the insertion unit 250 passing through the tissue surface and inserted into the tissue is provided in the handpiece 200. The insertion unit 250 of the present embodiment is composed of microneedles 320 (see FIG. 4) that are easily inserted into the tissue, and in addition to this, may have various structures such as a single needle structure or a catheter. The microneedle 320 (see FIG. 4) of the present embodiment may be a needle having a diameter in a range of several to several μm to several thousand μm, and preferably, use a needle having a diameter in a range of 10 to 1,000 μm.

If the insertion unit 250 is used to be repeatedly inserted into the tissue of the patient's body, a hygiene problem may occur. Thus, the insertion unit 250 of the present embodiment is provided at the tip 203 detachable from the end portion of the handpiece body 202, and the tip 203 may be used by replacing after the treatment.

In detail, a detachment protrusion 207 formed to protrude outward is formed on an outer wall of a base 301 (see FIG. 4) forming a bottom surface of the tip 203. In a recess portion 240 to which the tip 203 is coupled, a guide groove 241 guiding insertion of the detachment protrusion 207 and isolation prevention groove 242 for preventing the detachment protrusion 307 inserted along the guide groove 241 from being isolated are formed. In addition, the detachment protrusion 307 of the tip 203 is installed on the handpiece in a manner that the detachment protrusion 307 is guided along the guide groove 241 and fastened to the isolation prevention groove 242. However, a coupled structure of the handpiece body 202 and the tip 203 illustrated in FIG. 2 is an example of a structure in which the tip 203 is installed detachably from the handpiece body 202, and the coupled structure of the handpiece body 202 and the tip 203 may be modified in a various manner. Further, the tip 203 may be integrally formed with the handpiece body 202.

The insertion unit 250 constituting the plurality of microneedles 320 (see FIG. 4) is installed in the tip 203, and is installed detachably from the recess portion 240 provided in one end of the body of the handpiece 200. A plurality of holes (not illustrated) into which the above-described output terminal 211 may be optionally inserted are provided in a rear surface of the tip 203. Accordingly, as the above-described output terminal 211 is moved forward/backward, the plurality of microneedles 320 housed in the tip 203 may move forward/backward. In addition, when the tip 203 is installed in the recess portion 240, the microneedles 320 of the tip 203 may be electrically connected to an RF circuit in the handpiece 200 and the RF energy may be transferred to the inside of the tissue at the treatment position through the microneedles 320.

The detailed configuration of the handpiece and the tip may be implemented in various ways with reference to the configuration disclosed in Korean Patent No. 10-1300123.

FIG. 3 is a block diagram illustrating a main control system of the RF treatment device in FIG. 1. Hereinafter, a structure of controlling the RF treatment device according to the present embodiment will be described in detail with reference to FIG. 3.

A control unit 140 controls operations of various components of the main body 100 and the handpiece 200. As illustrated in FIG. 3, the control unit 140 may control an operation of the driving unit 210 of the handpiece to insert the insertion unit 250 into the tissue, to withdraw the insertion unit 250 from the tissue, or to adjust the insertion depth of the insertion unit 250.

In addition, the control unit 140 may control the RF generator 111 to adjust an on/off operation of an RF pulse and an RF pulse parameter. As a result, the RF treatment device 1 may provide an RF pulse having an appropriate parameter after inserting the microneedles into the tissue.

Further, the control unit 140 may control a cooling gas valve 112 to adjust a supply timing of the cooling gas and a supply amount of the cooling gas. The cooling gas valve 112 may be provided between the refrigerant tank and the second line 300b or may be provided in the handpiece 200.

A setting unit 120 may set details of the treatment by the user. In addition, the control unit 140 controls various components to perform treatment operations based on the setting of the user by the setting unit 120. The setting unit 120 may include the display unit 102 and/or the switch described above. The display unit 102 may display various options to the user and the user may set an option in a manner of selecting the displayed options.

The RF treatment device 1 further includes a memory unit 130 in which various data are stored. The control unit 140 may store information required for controlling the RF treatment device or retrieve the data stored in the memory unit 130 and utilize the data for the control.

Furthermore, the RF treatment device further includes a monitoring unit 260. The monitoring unit 260 monitors condition information on the tissue corresponding to the treatment position during the treatment. The monitoring unit 260 monitors a temperature of a tissue temperature and an impedance of an RF energy transfer path formed via the tissue, or monitors at least one of various information required for treatment, such as whether contact is made with a handpiece, a pressurized state, or the like.

For example, the monitoring unit 260 of the present embodiment may be provided on a path where the RF energy is transferred, and monitor the impedance of the path where the RF energy is transferred via the tissue. The monitoring unit may be provided on the RF transfer path in the handpiece or the RF transfer path in the main body. The monitoring unit 260 may monitor an impedance value measured by flowing a separate test current into the insertion unit 250, or monitor an impedance value measured while an RF pulse for treatment is transferred. In this case, since the measured impedance is changed depending on patient characteristics, condition change in tissue, or the like, it may be interpreted as an ‘impedance of tissue’ for convenience. In the present disclosure, the monitoring unit 260 may monitor the impedance of the tissue before the treatment or during the treatment, and adjust the details of the treatment based on the impedance of the tissue.

FIG. 4 is a cross-sectional view schematically illustrating an internal structure of a tip end portion of the handpiece in FIG. 2.

An RF transferring unit 310 in which the plurality of needles 320 are installed is provided in a tip 203. The plurality of needles 320 are fixedly installed to the RF transferring unit 310 in a matrix. The RF transferring unit 310 is formed with an electrical circuit connected to each of the plurality of needles 320. The electrical circuit formed on the RF transferring unit 310 is electrically connected to the output terminal 211 and allows the RF energy transferred through the output terminal 211 to transfer to the plurality of needles 320 by the RF transferring unit 310.

A tip end of the tip 203 may form a portion adjacent to or in contact with a patient's skin during the treatment, and a plurality of through-holes 302 through which the plurality of needles 320 appear are formed in the tip end of the tip 203.

At least one hole 303 is provided on a lower side of the tip 203 so as to pass through the output terminal 211. The output terminal 211 pressurizes the RF transferring unit 310 while linearly moving along the hole 303 when the driving unit 210 is operated. A rear surface of the RF transferring unit 310 is seated on supports 304 inside the tip 203, and a front surface of the RF transferring unit 310 is pressurized by elastic members 330 installed inside the tip 203.

When the output terminal 211 is moved to pressurize the RF transferring unit 310, the RF transferring unit 310 moves forward while being separated from the support 304, and the plurality of needles 320 are inserted into a skin tissue while protruding to front sides of the through-holes 302. In addition, when the output terminal 211 is moved backward by driving of the driving unit 210, the RF transferring unit 310 is moved backward due to a restoring force of the elastic member 330, and the plurality of needles 320 also return to the inside the tip 203. Although not separately illustrated, a separate guide member for guiding a path of moving the above-described RF transferring unit 310 may be further provided.

Although not specifically illustrated, when the tip 203 is installed on the handpiece body 202, the circuit of the RF transferring unit 310 may be electrically connected to the RF generator 111 of the main body 100. Alternatively, when the circuit of the RF transferring unit 310 is pressurized by the output terminal 211, the circuit may be electrically connected to the RF generator 111 in an optional manner (for example, an electrode is formed on an end portion of the output terminal 211, and electrically connected to the RF transferring unit 310 when pressurized).

Each needle 320 may be composed of a microneedle having a diameter of about 5 to 500 μm. The needle 320 is formed of a conductive material so as to transfer the RF energy. A portion other than the tip end portion of the surface of each needle is formed of an insulating material so as not to transfer the RF energy to the tissue. As a result, a portion of the tip end portion of each needle serves as an electrode so as to transfer the RF energy to the tissue only by the tip end portion. Accordingly, the RF energy may be optionally transferred to a portion where the end portion of the needle is positioned during the treatment.

Meanwhile, as illustrated in FIGS. 2 and 4, a cooling channel 205 passing through a side wall of the tip 203 is formed on one side of the tip 203. The cooling channel 205 is connected to the second line 300b so that the cooling gas supplied from the refrigerant tank flows into the tip 203.

The cooling gas flowed into the tip 203 through the cooling channel 205 is discharged through the through-hole 302 and cools the skin tissue with a rising temperature due to the RF energy transferred through the plurality of needles 320. The through-hole 302 is formed to have a larger diameter than the needle 320 so that the cooling gas flowed into the tip 203 may be discharged between the through-hole 302 and the needle 320 in a state in which the plurality of needles 320 pass through the through-holes 302, which is preferable.

A second line coupled port 204 extending the cooling channel 205 outward of the tip 203 may be formed on one side of the tip 203. An example of the coupled port 204 that facilitates the connection of the second line 300b and the tip 203 is illustrated, and it may be modified in various ways according to the embodiment. For example, the coupled port is formed on the side wall of the tip 203 in a recessed manner so that an end portion of the second line 300b is coupled to be fitted into the coupled port.

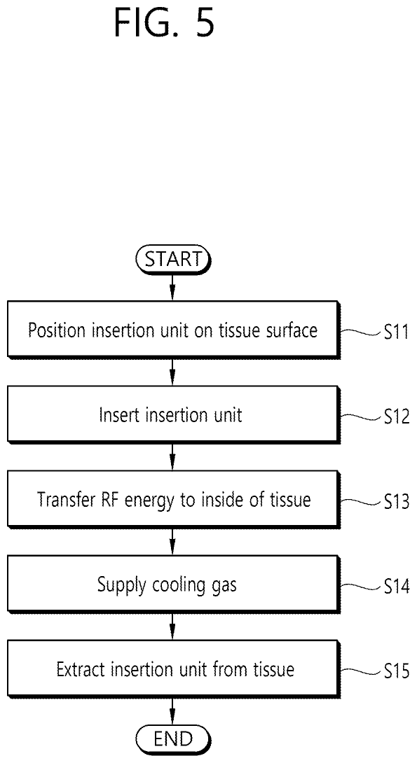

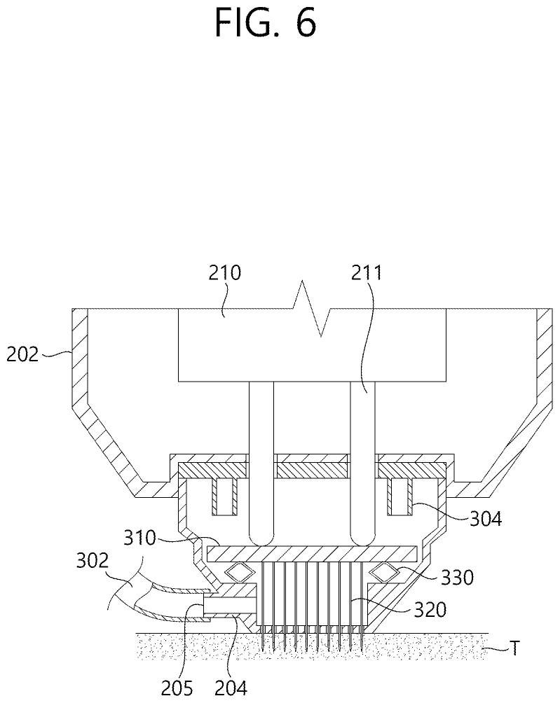

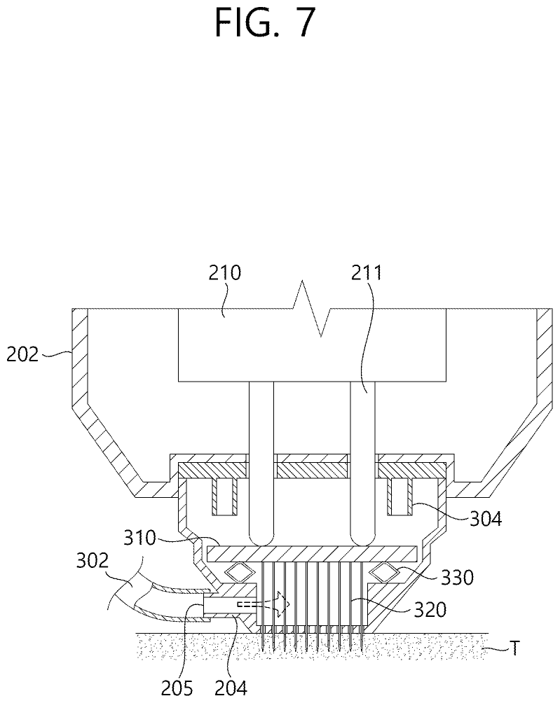

FIG. 5 is a flowchart illustrating a treatment method using the RF treatment device according to an embodiment of the present disclosure, and FIGS. 6 and 7 are views for explaining the treatment method using the RF treatment device according to an embodiment of the present disclosure. Hereinafter, an operation of the RF treatment device according to the present embodiment will be described with reference to FIGS. 4 to 7.

First, the insertion unit 250 of the treatment device is positioned on a surface of the tissue to be treated (S11). In detail, the tip end portion (tip 203) of the handpiece 200 having the insertion unit 250 built therein is positioned to be adjacent to or in contact with the surface of the tissue corresponding to the treatment position.

Subsequently, a step of inserting the insertion unit 250 into the tissue is performed (S12). The control unit 140 operates the driving unit 210 to move the insertion unit 250 forward. That is, by operating the driving unit 210, the control unit 140 controls the tip end portions of the plurality of needles 320 to pass through the through-holes 302, and then pass through a surface of a tissue T to be inserted into the tissue T as illustrated in FIG. 6.

Subsequently, a step of transferring the RF energy to the inside of the tissue is performed (S13). The control unit 140 controls the RF generator 111 to transfer the RF energy to the inside of the tissue T through the first line 300a, the output terminal 211, the RF transferring unit 310, and the plurality of needles 320.

Subsequently, a step of supplying the cooling gas is performed (S14). The control unit 140 controls the cooling gas valve 112 to flow the cooling gas from the refrigerant tank into the housing 201 of the handpiece 200 through the second line 300b and the cooling channel 205 as illustrated in FIG. 7. The cooling gas flowed into the housing 201 is sprayed onto the surface of the tissue T through the through-hole 302. When a temperature of the tissue T absorbing the RF energy rises, the cooling gas sprayed through the through-hole 302 cools the tissue T, thereby lowering the temperature.

Subsequently, when the treatment on the tissue T ends, a step of extracting the insertion unit 250 from the tissue is performed (S15). The control unit 140 operates the driving unit 210 to move the insertion unit 250 backward. That is, by operating the driving unit 210, the control unit 140 controls the tip end portions of the plurality of needles 320 to come out of the surface of the tissue, thereby completing the treatment.

FIG. 5 illustrates that each step is sequentially performed, but is not limited to this. It is possible to perform each step by changing the order thereof or to simultaneously perform a plurality of steps. For example, the step of supplying the cooling gas (S14) may be simultaneously performed with at least one of the step of inserting the insertion unit (S12) and the step of transferring the RF energy to the inside of the tissue (S13), or may be performed while the step of extracting the insertion unit from the tissue (S15) is being performed.

As described above, in the RF treatment device 1 and the treatment method according to the present embodiment, the cooling gas is supplied to a treatment site where the RF energy is supplied and cools the tissue with a rising temperature during the treatment process, such that it is possible to prevent a burn of the tissue and perform more effective treatment.

In particular, in the RF treatment device 1 and the treatment method according to the present embodiment, the cooling gas is sprayed through the through-hole 302 of the handpiece 200 positioned to be closest to the treatment site, such that it is possible to efficiently cool the treatment site.

Hereinafter, handpieces according to another embodiment of the present disclosure will be described. For convenience of explanation, the components similar to those of the above-described embodiment are denoted by the same reference numerals, and a description thereof will be omitted.

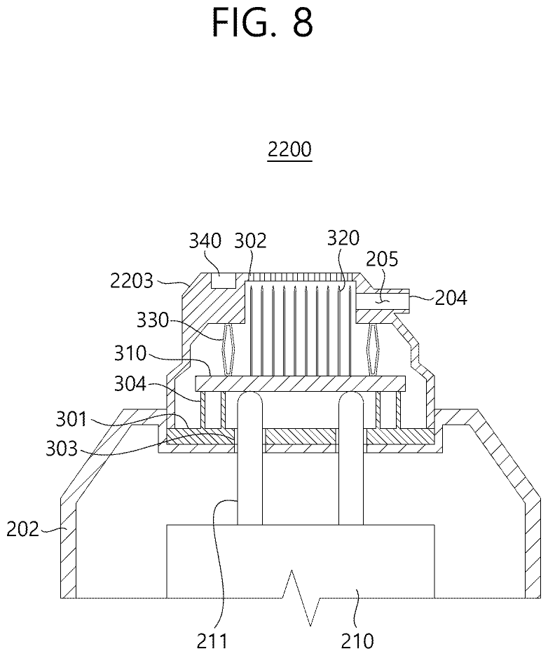

FIG. 8 is a cross-sectional view schematically illustrating an internal structure of a tip end portion of the handpiece according to another embodiment of the present disclosure.

As illustrated in FIG. 8, a handpiece 2200 according to another embodiment of the present disclosure further includes a temperature measurement unit 340 as compared with the handpiece 200 according to the above-described embodiment.

The temperature measurement unit 340 measures a temperature of the tissue positioned to be adjacent to or in contact with a tip end of a tip 2203. The temperature measurement unit 340 may include a contact type (electronic) temperature measurement sensor or a non-contact type (optical) temperature measurement sensor. In a case where the temperature measurement unit 340 includes a contact type temperature measurement sensor, the treatment on the tissue may be performed while the tip end of the tip 2203 is in contact with a surface of the tissue. In a case where the temperature measurement unit 340 includes a non-contact type temperature measurement sensor, the treatment on the tissue may be performed while the tip end of the tip 2203 is adjacent to the surface of the tissue.

FIG. 8 illustrates an example in which the temperature measurement unit 340 is provided on a tip end of the tip 2203, but the temperature measurement unit 340 may be provided on other portions of the tip 2203 or the handpiece body 202 so long as a temperature of the tissue where the treatment is performed may be measured.

The temperature measurement unit 340 is connected to the control unit 140 and transmits a temperature measurement result to the control unit 140. The control unit 140 enables control of a supply state of the colling gas by controlling the cooling gas valve 112 based on the temperature measurement result of the temperature measurement unit 340.

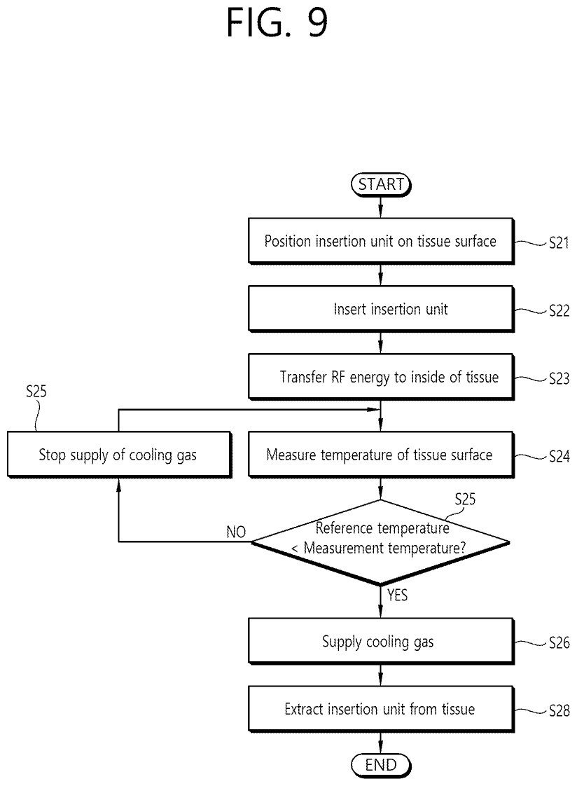

FIG. 9 is a flowchart illustrating a treatment method using the handpiece in FIG. 8.

As illustrated in FIG. 9, the treatment method using the RF treatment device according to the present embodiment also includes a step of positioning the insertion unit on the surface of the tissue to be treated (S21), a step of inserting the insertion unit into the tissue (S22), and a step of transferring the RF energy to the inside of the tissue (S23). This is the same as or similar to steps S11, S12, and S13 described in the treatment method using the RF treatment device 1 of the above-described embodiment, and thus, the detailed description thereof will be omitted.

After the RF energy is transferred to the inside of the tissue, a step of measuring a temperature of the tissue surface is performed (S24). The temperature measurement unit 340 measures the temperature of the tissue surface and transmits it to the control unit 140. The temperature measurement unit 340 measures the temperature of the tissue surface and transmits it to the control unit 140 even during the treatment.

Subsequently, a step of comparing a measurement temperature with a reference temperature is performed (S25). The control unit 140 compares the temperature of the tissue surface received from the temperature measurement unit 340 with the reference temperature. The reference temperature may be a temperature value input in advance or a temperature value automatically calculated by the control unit 140.

FIG. 9 illustrates an example in which it is determined whether the measurement temperature exceeds the reference temperature, but is not limited to this. It may be set to determine whether the measurement temperature is equal to or higher than the reference temperature or it may be set to determine whether the measurement temperature is lower than (or equal to or lower than) the reference temperature. Alternatively, the reference temperature includes a cooling start temperature and a cooling stop temperature, and it may be set to compare the measurement temperature with a cooling start temperature and a cooling stop temperature.

For convenience of explanation, hereinafter, determination, by the control unit 140, of whether the measurement temperature exceeds the reference temperature will be described by way of example.

When the measurement temperature exceeds the reference temperature, a step of supplying the cooling gas is performed (S26). When the measurement temperature exceeds the reference temperature, the control unit 140 controls the cooling gas valve 112 so that the cooling gas is supplied into the handpiece 2200 through the cooling channel 205 and sprayed onto the surface of the tissue where the treatment is being performed through the through-hole 302. When the reference temperature includes the cooling start temperature and the cooling stop temperature, the control unit 140 may compare the cooling start temperature of the reference temperature with the measurement temperature in step S26.

When the measurement temperature does not exceed the reference temperature, a step of stopping supply of the cooling gas is performed (S27). Then, step S25 is performed again.

Step S27 includes not only the stopping of supply the cooling gas supplied to the surface of the tissue, but also maintaining a state in which the cooling gas is not supplied. When the measurement temperature does not exceed the reference temperature, the control unit 140 controls the cooling gas valve 112 so that the cooling gas is not supplied through the second line 300b. When the reference temperature includes the cooling start temperature and the cooling stop temperature, the control unit 140 may compare the cooling stop temperature of the reference temperature with the measurement temperature in step S27.

Subsequently, when the treatment on the tissue ends, a step of extracting the insertion unit 250 from the tissue is performed (S28). Step S28 is the same as or similar to step S15 described above, and thus the detailed description thereof will be omitted.

FIG. 9 illustrates that each step is sequentially performed, but is not limited to this. It is possible to perform each step by changing the order thereof or to simultaneously perform a plurality of steps. For example, the step of measuring the temperature of the tissue surface (S24), the step of comparing the measurement temperature with the reference temperature (S25), the step of supplying the cooling gas (S26), or the like are simultaneously performed with at least one of the step of inserting the insertion unit (S22) and the step of transferring the RF energy to the inside of the tissue (S23).

In the RF treatment device and the treatment method using the handpiece 2200 according to the present embodiment, the temperature of the tissue surface is measured to control supply of the cooling gas corresponding to the temperature of the tissue surface, such that it is possible to perform more effective treatment. In particular, it is possible to prevent frostbite on the tissue due to oversupply of the cooling gas.

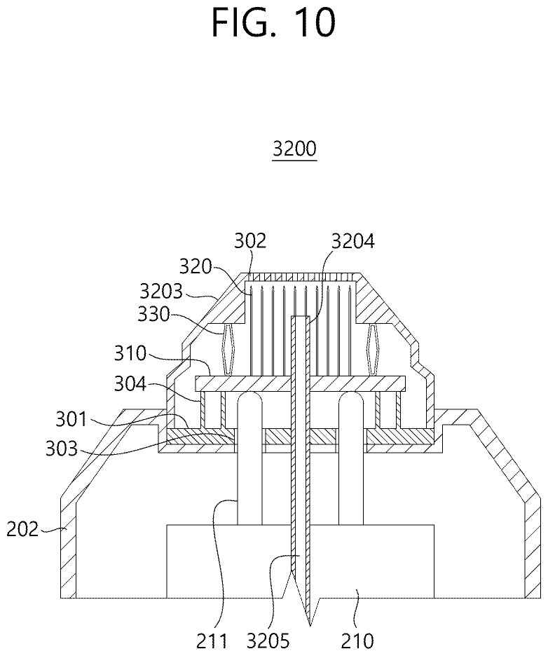

FIG. 10 is a cross-sectional view schematically illustrating an internal structure of a tip end portion of the handpiece according to another embodiment.

As illustrated in FIG. 10, a handpiece 3200 according to another embodiment has a different position of a cooling channel 3205 as compared with the handpiece 200 according to the above-described embodiment.

In the handpieces 200 and 2200 according to the above-described embodiments, the cooling channel 205 is formed through the tips 203 and 2203, whereas in the handpiece 3200 according to the present embodiment, the cooling channel 3205 is formed on a side of the handpiece body 202.

In more detail, the cooling channel 3205 is formed by a cooling pipe 3204 exposed to the tip end of the handpiece body 202 by the driving unit 210. The cooling pipe 3204 may be fixedly installed in the handpiece body 202 regardless of movement of the output terminal 211 by the driving unit 210, but is not limited to this. According to the embodiment, the driving unit 210 may integrally move the output terminal 211 with the cooling pipe 3204 when moving the output terminal 211.

In a case of the present embodiment, the second line 300b transferring the cooling gas through the cooling channel 3205 may be connected to the cooling channel 3205 together with the first line 300a through a rear end of the handpiece body 202. To this end, the cooling pipe 3204 may be connected to the second line 300b by being exposed to the rear end of the handpiece body 202. However, according to the embodiment, the cooling pipe 3204 and the second line 300b may be connected to the inside of the handpiece body 202, or the cooling pipe 3204 and the second line 300b may be formed integrally.

A cooling pipe entrance hole (no reference numeral assigned) may be formed in the base 301 of the tip 3203 and the RF transferring unit 310 so that the cooling pipe 3204 exposed to the tip end of the handpiece body 202 enters the inside of the tip 3203.

A portion of the cooling pipe 3204 exposed to the tip end of the handpiece body 202 may be formed of a rigid material so that the cooling pipe 3204 exposed to the tip end of the handpiece body 202 easily enters the cooling pipe entrance hole when the tip 3203 is coupled to the handpiece 202.

As illustrated in FIG. 10, an end portion of the cooling pipe 3204 may be positioned between the RF transferring unit 310 and the tip end of the tip 3203 while the tip 3203 is coupled to the handpiece body 202. Alternatively, according to the embodiment, the end portion of the cooling pipe 3204 may be positioned on the RF transferring unit 310.

In the handpiece 3200 according to the present embodiment, the cooling channel 3205 is also formed through the cooling pipe 3204 to the inside of a tip 3203 and the cooling gas is spayed through the through-hole 302 of the handpiece 2200, and thus the treatment site may be effectively cooled.

Although not illustrated, according to another embodiment, the end portion of the cooling pipe 3204 may be positioned between the base 301 and the RF transferring unit 310.

In this case, the cooling gas supplied between the base 301 and the RF transferring unit 310 may be released from the outside of the tip 3203 through the through-hole 302 in a state where the driving unit 210 moves the output terminal 211 forward so that the RF transferring unit 310 is separated from the support 304.

The support 304 may be formed in a ring shape surrounding a periphery of the cooling pipe 3204. In a state where the RF transferring unit 310 is in contact with the support 304, the cooling gas may not be easily leaked from a space partitioned by the RF transferring unit 310 and the support 304. That is, the space partitioned by the RF transferring unit 310 and the support 304 may be a closed space.

For example, the space partitioned by the RF transferring unit 310 and the support 304 may be formed as a closed space by providing a sealing member at a portion of an upper end of the support 304 or a lower surface of the RF transferring unit 310 in contact with the upper end of the support 304.

With the configuration described above, the cooling gas flowed into the tip 3203 through the cooling channel 3205 may be discharged through the through-hole 302 only in a state where the plurality of needles 320 are moved to be inserted into the tissue. Therefore, in a state where the RF transferring unit 310 and the plurality of needles 320 are moved toward the through-hole 302 for treatment, the cooling gas is discharged through the through-hole 302. In a state where the RF transferring unit 310 is in contact with the upper end of the support 304 when the treatment is not performed, the cooling gas may not be discharged through the through-hole 302.

Although not illustrated, the handpiece 3200 according to the present embodiment may further include the temperature measurement unit 340 (see FIG. 8). The control of the cooling gas using the temperature measurement unit 340 has been described, and thus the detailed description thereof will be omitted.

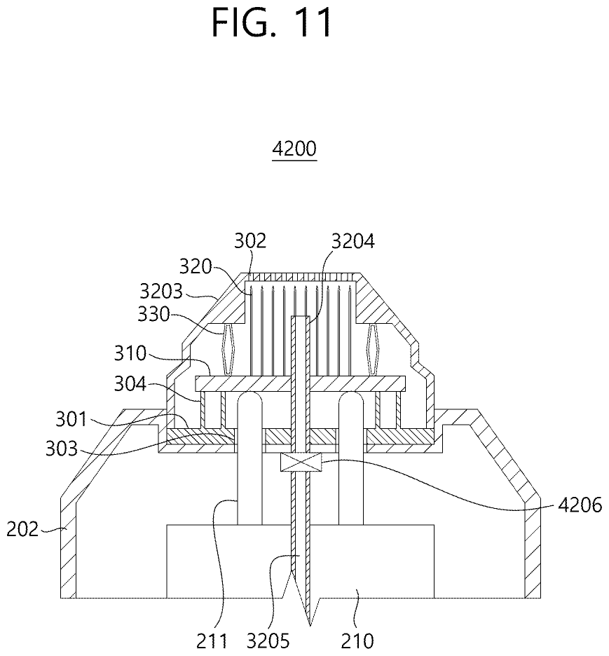

FIG. 11 is a cross-sectional view schematically illustrating an internal structure of a tip end portion of the handpiece according to another embodiment.

As illustrated in FIG. 11, a handpiece 4200 according to another embodiment further includes a valve 4206 on the cooling channel 3205 as compared with the handpiece 3200 according to the above-described embodiment.

The valve 4206 is a valve for opening and closing the cooling channel 3205, and the valve 4206 may open and close the cooling channel 3205 in conjunction with the movement of the plurality of needles 320 and the RF transferring unit 310.

For example, the valve 4206 may open and close the cooling channel 3205 in conjunction with the movement of the output terminal 211. In this case, when the output terminal 211 is moved forward through the through-hole 302 and maintained the state of being moved forward, the valve 4206 opens the cooling channel 3205 to spray the cooling gas to the tissue through the through-hole 302. When the output terminal 211 is moved backward and maintained in the state of being moved backward, the valve 4206 closes the cooling channel 3205 to block the cooling gas being supplied into the tip 3203.

The valve 4206 may switch open and close in synchronization with the movement of the output terminal 211 in a mechanical manner, or may switch open and close by a control signal in an electrical manner.

FIG. 11 illustrates an example in which the valve 4206 is provided in the handpiece body 202, but the valve 4206 may be provided in the tip 3203. In this case, the valve 4206 may open and close the cooling channel 3205 in conjunction with the movement of the output terminal 211 or the RF transferring unit 310.

The handpiece 4200 according to the present embodiment blocks spray of the cooling gas into the tip 3203 in a state where the treatment is not performed, and after the RF transferring unit 310 and the plurality of needles 320 are moved for treatment, the cooling gas may be supplied into the tip 3203.

Hereinabove, the treatment device performing the treatment by transferring the RF energy to the skin tissue has been mainly described. However, this is merely an example, and may be applied to a treatment device focused on other tissue rather than the skin tissue. Furthermore, the treatment device may be applied to various treatment devices, such as a treatment device performing treatment using a method of transferring energy such as RF, laser, or ultrasonic wave, a treatment device performing treatment using a method of transferring a treating substance (for example, a drug, an anesthetic, and a stem cell), in addition to a treatment device performing treatment using a method of transferring RF energy.

Moreover, the treatment device including the main body and the handpiece has been mainly described, but is not limited thereto, and may be applied to a treatment device configured in a single module form of the handpiece.

It is evident to those skilled in the art that the present disclosure may be materialized in other specific forms without departing from the essential characteristics of the present disclosure. Accordingly, the detailed description should not be construed as being limitative from all aspects, but should be construed as being illustrative. The scope of the present disclosure should be determined by reasonable analysis of the attached claims, and all changes within the equivalent range of the present disclosure are included in the scope of the present disclosure.

### Mode for Carrying Out Disclosure

According to an embodiment of the present disclosure, a handpiece for treatment includes: a housing having at least one through-hole formed in a tip end thereof; an insertion unit passing through a tissue surface and inserted into a tissue in a state in which at least a portion thereof is exposed through the through-hole; and a cooling channel providing a cooling gas supplied to the tissue surface through the through-hole.

The cooling channel may be formed to pass through one side of the housing, and the cooling gas may be provided in the housing.

The insertion unit may include an RF transferring unit transferring RF energy, and the cooling channel may be formed to pass through the RF transferring unit.

The handpiece for treatment may further include a driving unit moving the insertion unit so that at least a portion of the insertion unit passes through the through-hole.

The cooling channel may be formed to pass through the driving unit and supply the cooling gas to the through-hole.

The cooling gas may be supplied through the cooling channel after the driving unit moves the insertion unit.

The cooling channel may be opened and closed in conjunction with a movement of the insertion unit by the driving unit.

The handpiece for treatment may further include a temperature measurement unit measuring a temperature of the tissue surface.

The cooling gas may be supplied based on the temperature of the tissue surface measured by the temperature measurement unit.

The cooling gas may include at least one of cryogen and air.

According to an embodiment of the present disclosure, a method of controlling a treatment device includes: positioning an insertion unit on a tissue surface; inserting at least a portion of the insertion unit into a tissue by passing through the tissue surface; and supplying a cooling gas to the tissue surface.

The method may further include transferring RF energy to an inside of the tissue by the insertion unit, wherein the supplying of the cooling gas may be performed after the transferring of the RF energy.

The method may further include measuring a temperature of the tissue surface, wherein the supplying of the cooling gas may be performed when the temperature of the tissue surface is equal to or higher than or exceeds a predetermined cooling start temperature.

The method may further include stopping the supply of the cooling gas, wherein the stopping of supply of the cooling gas may be performed when the temperature of the tissue surface is equal to or lower than or lower than a predetermined cooling stop temperature.

In the inserting of at least a portion of the insertion unit into the tissue, the insertion unit may be moved toward the tissue, and the supplying of the cooling gas may be performed after the insertion unit is moved toward the tissue surface.

According to an embodiment of the present disclosure, an RF treatment device includes: a main body including an RF generator and a refrigerant tank; and a handpiece connected to the main body, wherein the handpiece includes a housing having at least one through-hole formed in a tip end thereof; an insertion unit passing through a tissue surface and inserted into a tissue in a state in which at least a portion thereof is exposed through the through-hole, and applying RF energy transferred from the RF generator to an inside of the tissue; and a cooling channel providing a cooling gas transferred from the refrigerant tank to the tissue surface through the through-hole.

A first line and a second line connecting the main body and the handpiece may be provided, the first line may transfer the RF energy from the RF generator to the handpiece, and the second line may transfer the cooling gas from the refrigerant tank to the cooling channel.

The handpiece may further include an RF transferring unit connected to the first line and transferring the RF energy, and the cooling channel may be formed to pass through the RF transferring unit.

The first line and the second line may be each connected to the RF transferring unit and the cooling channel through a rear end of the housing.

The first line may transfer the RF energy to the insertion unit through the rear end of the housing, and the second line may be connected to the cooling channel through one side of the housing.

1. A handpiece for treatment, comprising:
a housing having at least one through-hole formed in a tip end thereof;
an insertion unit passing through a tissue surface and inserted into a tissue in a state in which at least a portion thereof is exposed through the through-hole; and
a cooling channel providing a cooling gas supplied to the tissue surface through the through-hole.

2. The handpiece of claim 1, wherein the cooling channel is formed to pass through one side of the housing, and the cooling gas is provided in the housing.

3. The handpiece of claim 1, wherein the insertion unit includes an RF transferring unit transferring RF energy, and
the cooling channel is formed to pass through the RF transferring unit.

4. The handpiece of claim 1, further comprising a driving unit moving the insertion unit so that at least a portion of the insertion unit passes through the through-hole.

5. The handpiece of claim 4, wherein the cooling channel is formed to pass through the driving unit and supply the cooling gas toward the through-hole.

6. The handpiece of claim 4, wherein the cooling gas is supplied through the cooling channel after the driving unit moves the insertion unit.

7. The handpiece of claim 6, wherein the cooling channel is opened and closed in conjunction with a movement of the insertion unit by the driving unit.

8. The handpiece of claim 1, further comprising a temperature measurement unit measuring a temperature of the tissue surface.

9. The handpiece of claim 8, wherein the cooling gas is supplied based on the temperature of the tissue surface measured by the temperature measurement unit.

10. The handpiece of claim 1, wherein the cooling gas includes at least one of cryogen and air.

11. A method of controlling a treatment device, the method comprising:
positioning an insertion unit on a tissue surface;
inserting at least a portion of the insertion unit into a tissue by passing through the tissue surface; and
supplying a cooling gas to the tissue surface.

12. The method of claim 11, further comprising transferring RF energy to an inside of the tissue by the insertion unit,
wherein the supplying of the cooling gas is performed after the transferring of the RF energy.

13. The method of claim 11, further comprising measuring a temperature of the tissue surface,
wherein the supplying of the cooling gas is performed when the temperature of the tissue surface is equal to or higher than or exceeds a predetermined cooling start temperature.

14. The method of claim 13, further comprising stopping supply of the cooling gas,
wherein the stopping of supply of the cooling gas is performed when the temperature of the tissue surface is equal to or lower than or lower than a predetermined cooling stop temperature.

15. The method of claim 11, wherein in the inserting of at least a portion of the insertion unit into the tissue, the insertion unit is moved toward the tissue, and
the supplying of the cooling gas is performed after the insertion unit is moved toward the tissue surface.

16. An RF treatment device comprising: a main body including an RF generator and a refrigerant tank; and a handpiece connected to the main body,
wherein the handpiece includes:
a housing having at least one through-hole formed in a tip end thereof;
an insertion unit passing through a tissue surface and inserted into a tissue in a state in which at least a portion thereof is exposed through the through-hole, and applying RF energy transferred from the RF generator to an inside of the tissue; and
a cooling channel providing a cooling gas transferred from the refrigerant tank to the tissue surface through the through-hole.

17. The RF treatment device of claim 16, wherein a first line and a second line connecting the main body and the handpiece are provided,
the first line transfers the RF energy from the RF generator to the handpiece, and
the second line transfers the cooling gas from the refrigerant tank to the cooling channel.

18. The RF treatment device of claim 17, wherein the handpiece further includes:
an RF transferring unit connected to the first line and transferring the RF energy; and
the cooling channel is formed to pass through the RF transferring unit.

19. The RF treatment device of claim 18, wherein the first line and the second line are each connected to the RF transferring unit and the cooling channel through a rear end of the housing.

20. The RF treatment device of claim 17, wherein the first line transfers the RF energy to the insertion unit through the rear end of the housing, and
the second line is connected to the cooling channel through one side of the housing.
